(19) United States
(12) Patent Application Publication
(10) Pub. No.: US 2024/0398383 A1
(43) Pub. Date: Dec. 5, 2024

(54) ULTRASOUND DIAGNOSTIC SYSTEM AND EXTRACTION APPARATUS

(71) Applicant: FUJIFILM Healthcare Corporation, Chiba (JP)

(72) Inventors: MASAMI EBIKUSA, Chiba (JP); Takanori Hirai, Chiba (JP)

(73) Assignee: FUJIFILM Healthcare Corporation, Chiba (JP)

(21) Appl. No.: 18/674,892

(22) Filed: May 26, 2024

(30) Foreign Application Priority Data
May 29, 2023 (JP) 2023-087502

(51) Int. Cl. A61B 8/08; A61B 8/00

(52) U.S. Cl. CPC A61B 8/5207; A61B 8/461

(57) ABSTRACT

An ultrasound diagnostic apparatus sends a search request including reference frame data to an image server. In the image server, in a case where the volume data is specified, the volume data is sent to an extraction server. The extraction server extracts fitting frame data that fits the reference frame data from the volume data. A thumbnail image representing the fitting frame data is displayed in the ultrasound diagnostic apparatus.

### CROSS-REFERENCE TO RELATED APPLICATION

This application claims the priority benefits of Japanese application no. 2023-087502, filed on May 29, 2023. The entirety of the above-mentioned patent application is hereby incorporated by reference herein and made a part of this specification.

### BACKGROUND

### 1. Technical Field

The present disclosure relates to an ultrasound diagnostic system and an extraction apparatus, and particularly relates to a technique of processing volume data stored in a server.

### 2. Description of the Related Art

An ultrasound diagnostic apparatus is used in an ultrasound examination of a subject. In general, the ultrasound diagnostic apparatus is connected to a server, typically a Digital Imaging and Communications in Medicine (DICOM) server, via a network. A large number of pieces of data are stored in the server. A search is executed in the server in response to a search request and a transmit request from the ultrasound diagnostic apparatus, and the data specified by the search is transmitted from the server to the ultrasound diagnostic apparatus.

A data group stored in the server includes a plurality of pieces of volume data. Examples of such volume data include volume data acquired by an ultrasound diagnostic apparatus (ultrasound volume data), volume data acquired by a magnetic resonance imaging (MRI) apparatus (MR volume data), and volume data acquired by a computed tomography (CT) apparatus (X-ray CT apparatus) (CT volume data). With regard to a structure of the volume data, there is aggregate-type volume data configured as an aggregate of a plurality of pieces of frame data (plurality of pieces of slice data) and continuous-type volume data configured as one data block having three-dimensional continuity.

In a case where specific volume data to be referred to is the aggregate-type volume data, in a case where a transmit request including a frame number (usually, a center frame number) is sent from the ultrasound diagnostic apparatus to the server, frame data specified by the frame number is transmitted from the server to the ultrasound diagnostic apparatus. In the ultrasound diagnostic apparatus, a thumbnail image is generated based on the acquired frame data, and the thumbnail image is displayed.

JP2009-22626A discloses an ultrasound diagnostic apparatus including a DICOM server. The ultrasound diagnostic apparatus has a function of displaying a current ultrasound image and a past ultrasound image in juxtaposition. JP2009-22626A does not disclose a technique of searching for desired frame data from volume data.

### SUMMARY

From the viewpoint of supporting an ultrasound examination of a subject, it is desired to promote use of a past volume data by causing an examiner to recognize presence of the past volume data acquired from the subject and stored in a server during the ultrasound examination of the subject.

In order to satisfy the request, in a case where the server is caused to search for the past volume data of the subject and the server is caused to send all the past volume data to the ultrasound diagnostic apparatus, a burden on a network is increased. It is desired that only data representing a portion that the examiner would like to see in the past volume data is sent to the ultrasound diagnostic apparatus.

An object of the present disclosure is to enable an examiner to recognize presence of volume data stored in a server without imposing a large burden on a network during an ultrasound examination. Alternatively, an object of the present disclosure is to display an image that the examiner would like to see as an image representing volume data in the ultrasound diagnostic apparatus.

An ultrasound diagnostic system according to an aspect of the present disclosure includes an ultrasound diagnostic apparatus configured to form an ultrasound image based on reception data obtained from a subject; and an extraction apparatus configured to extract, in a case where past volume data obtained from the subject is specified from a data group stored in a server, fitting frame data that fits reference frame data sent from the ultrasound diagnostic apparatus from the past volume data, in which the ultrasound diagnostic apparatus displays an image representing the fitting frame data together with the ultrasound image during an ultrasound examination of the subject.

An extraction apparatus according to an aspect of the present disclosure includes means for receiving reference frame data sent from an ultrasound diagnostic apparatus; means for extracting, in a case where past volume data obtained from a subject is specified from a data group stored in a server, fitting frame data that fits the reference frame data from the past volume data; and means for transmitting the fitting frame data to the ultrasound diagnostic apparatus, in which an image representing the fitting frame data is displayed together with an ultrasound image during an ultrasound examination of the subject, in the ultrasound diagnostic apparatus.

According to the present disclosure, the examiner can recognize the presence of the volume data stored in the server without imposing a large burden on the network during the ultrasound examination. Alternatively, according to the present disclosure, the image that the examiner would like to see is displayed as the image representing the volume data in the ultrasound diagnostic apparatus.

### BRIEF DESCRIPTION OF THE DRAWINGS

FIG. 1 is a block diagram showing an ultrasound diagnostic system according to a first embodiment.

FIG. 2 is a diagram showing an example of extraction processing (secondary extraction processing).

FIG. 3 is a flowchart showing an operation of the ultrasound diagnostic system according to the first embodiment.

FIG. 4 is a diagram showing an example of a screen image.

FIG. 5 is a diagram showing reference frame data.

FIG. 6 is a diagram showing an operation of an ultrasound diagnostic system according to a second embodiment.

FIG. 7 is a diagram showing switching of thumbnail images according to the second embodiment.

FIG. 8 is a block diagram showing an ultrasound diagnostic system according to a third embodiment.

### DESCRIPTION OF THE EMBODIMENTS

Hereinafter, embodiments will be described with reference to the accompanying drawings.

### (1) Outline of Embodiments

An ultrasound diagnostic system according to the embodiments includes an ultrasound diagnostic apparatus and an extraction apparatus. The ultrasound diagnostic apparatus forms an ultrasound image based on reception data obtained from a subject. The extraction apparatus extracts fitting frame data that fits reference frame data sent from the ultrasound diagnostic apparatus from past volume data in a case where the past volume data obtained from the subject is specified from a data group stored in a server. The ultrasound diagnostic apparatus displays an image representing the fitting frame data together with the ultrasound image during an ultrasound examination of the subject.

With the above configuration, an image representing the past volume data acquired from the subject is displayed during the ultrasound examination of the subject. Therefore, the examiner can recognize that the past volume data is present in the server through observation of such an image. The image representing the past volume data is an image representing the fitting frame data that fits the reference frame data. Therefore, a possibility that an image that can support a current ultrasound examination is displayed can be increased on the premise of appropriate selection of the reference frame data.

The image representing the fitting frame data is, for example, a tomographic image or a thumbnail image. In a case where acquisition of the fitting frame data and generation of the image representing the fitting frame data are executed as the background processing during the ultrasound examination of the subject, a burden on the examiner does not occur. With the above configuration, since not all but only a part of the past volume data is transmitted, a load on a network is reduced. The advantage is further increased in a case where a plurality of pieces of the past volume data are present in the server. At a point in time at which it is necessary to acquire all the past volume data, the past volume data is transmitted from the server to the ultrasound diagnostic apparatus.

In the embodiments, the extraction apparatus is an information processing apparatus connected to the ultrasound diagnostic apparatus via a network. The extraction apparatus may be incorporated into the server. An external information processing apparatus that is physically coupled to the ultrasound diagnostic apparatus may function as the extraction apparatus. The past volume data is MR volume data, CT volume data, or ultrasound volume data. The past volume data may be other volume data, such as X-ray mammography volume data. According to the configuration according to the embodiments, it is possible to promote use of past volume data acquired by a medical apparatus of a type different from that of the ultrasound diagnostic apparatus.

In the embodiments, the extraction apparatus compares the reference frame data with a plurality of candidate frame data included in the past volume data to extract specific candidate frame data as the fitting frame data. In a case of comparing two pieces of frame data, various well-known techniques such as a correlation calculation can be used. In general, cross sections for cutting out the candidate frame data are sequentially set for the past volume data at various positions and various angles. In a case where the volume data is composed of a plurality of pieces of frame data, only the plurality of pieces of frame data may be a plurality pieces of candidate frame data.

In the embodiments, the ultrasound diagnostic apparatus includes a thumbnail image generation unit that generates a thumbnail image as the image representing the fitting frame data, based on the fitting frame data. In general, the thumbnail image is generated by low-resolution processing on the fitting frame data. The thumbnail image can be described as a reduced tomographic image. In general, through a selection operation of a thumbnail image, a tomographic image (a tomographic image that has not been subjected to low-resolution processing) representing the corresponding frame data associated with the thumbnail image is displayed.

In the embodiments, the ultrasound diagnostic apparatus displays a screen image including the ultrasound image. The above-described thumbnail image is a first thumbnail image. The thumbnail image generation unit further generates a second thumbnail image representing the reception data obtained by the ultrasound diagnostic apparatus. The screen image includes an external data display area in which the first thumbnail image is displayed, and an internal data display area in which the second thumbnail image is displayed.

With the above configuration, the examiner can recognize local data (internal data) stored in the ultrasound diagnostic apparatus and can recognize remote data (external data) stored in the server. Therefore, acquisition, reference, and use of the external data can be promoted while distinguishing the external data from the internal data.

In the embodiments, the above-described extraction apparatus is a secondary extraction apparatus. The above-described thumbnail image is a secondary thumbnail image. The ultrasound diagnostic system further includes a primary extraction apparatus that extracts designated frame data from the past volume data based on designated frame identification information sent from the ultrasound diagnostic apparatus. The thumbnail image generation unit further generates a primary thumbnail image as an image representing the designated frame data, based on the designated frame data. The ultrasound diagnostic apparatus displays the primary thumbnail image and the secondary thumbnail image.

A designated frame is usually a frame automatically designated by the ultrasound diagnostic apparatus. In a case where it takes time to display the secondary thumbnail image, the primary thumbnail image may be provisionally displayed in advance. The primary thumbnail image and the secondary thumbnail image can be displayed at the same time or sequentially.

In the embodiments, the ultrasound diagnostic apparatus displays the primary thumbnail image first, and then displays the secondary thumbnail image instead of the primary thumbnail image. With this configuration, the examiner can recognize the presence of the past volume data at an early stage, and it is possible to provide the image that the examiner would like to see to the examiner. In a case where a plurality of secondary thumbnail images representing the plurality of pieces of volume data are displayed, the plurality of secondary thumbnail images may be sequentially displayed in a generation order thereof, or may be displayed at the same time at a certain timing.

In the embodiments, the ultrasound diagnostic apparatus displays identification information for identifying the primary thumbnail image and the secondary thumbnail image. With this configuration, the examiner can recognize properties of the frame data corresponding to the thumbnail image.

In the embodiments, the extraction apparatus calculates a fitting score in a case of comparing the reference frame data with each piece of candidate frame data. The ultrasound diagnostic apparatus displays information indicating the fitting score corresponding to the fitting frame data together with the image representing the fitting frame data. With this configuration, the examiner can recognize a possibility that a cross section corresponding to the fitting frame data is a cross section that the examiner would like to see, with reference to the information indicating the fitting score.

In the embodiments, the reference frame data is one of past tomographic image data registered in the ultrasound diagnostic apparatus, current tomographic image data acquired in the current ultrasound examination on the subject, or pseudo tomographic image data cut out from a three-dimensional tissue model.

The reference frame data is automatically designated or designated by the examiner. For example, a plurality of pieces of reference frame data associated with a plurality of processes constituting an ultrasound examination protocol may be registered in advance. With such a configuration, a plurality of pieces of the reference frame data are automatically sequentially selected and a plurality of the thumbnail images are automatically sequentially displayed as the ultrasound examination protocol progresses.

The extraction apparatus according to the embodiments includes a reception unit, an extraction unit, and a transmission unit. The reception unit receives the reference frame data sent from the ultrasound diagnostic apparatus. The extraction unit extracts the fitting frame data that fits the reference frame data from the past volume data in a case where the past volume data obtained from the subject is specified from the data group stored in the server. The transmission unit transmits the fitting frame data to the ultrasound diagnostic apparatus. An image representing the fitting frame data is displayed together with the ultrasound image during the ultrasound examination of the subject, in the ultrasound diagnostic apparatus.

Each of the above-described units is realized as a function of hardware or as a function of software. In the latter case, a program that executes the above-described series of pieces of processing is installed in the information processing apparatus via a network or a portable storage medium. The information processing apparatus includes a non-transitory storage medium in which the program is stored. In addition, the information processing apparatus includes a processor that executes the program.

### (2) Details of Embodiments

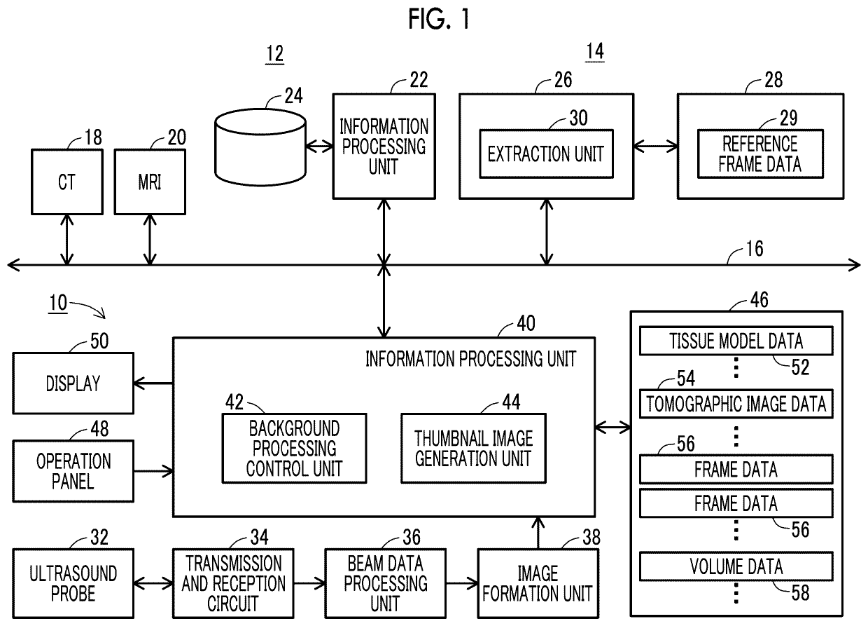

FIG. 1 shows an ultrasound diagnostic system according to a first embodiment. This ultrasound diagnostic system is provided in, for example, a medical institution such as a hospital. The ultrasound diagnostic system is actually a medical system for supporting an examination (and treatment) of a subject.

The ultrasound diagnostic system includes an ultrasound diagnostic apparatus 10, an image server 12, and an extraction server 14. The ultrasound diagnostic apparatus 10 is used in the ultrasound examination of the subject. The image server 12 is, for example, a DICOM server that stores and provides a medical image. The extraction server 14 is an apparatus that extracts frame data from volume data, as will be described later.

The ultrasound diagnostic system has a network 16. The network 16 is, for example, a local area network (LAN). The network 16 may include the Internet. The ultrasound diagnostic apparatus 10, the image server 12, and the extraction server 14 are connected to the network 16. A CT apparatus 18 and an MRI apparatus 20 are also connected to the network 16. In addition, another ultrasound diagnostic apparatus (not shown) is also connected to the network 16.

The image server 12 is configured by an information processing apparatus such as a computer. The image server 12 includes an information processing unit 22 and a storage unit 24. The information processing unit 22 includes a processor. The storage unit 24 is configured by a semiconductor memory, a hard disk, or the like. The storage unit 24 stores a data group consisting of various kinds of data acquired by the ultrasound diagnostic apparatus 10, the CT apparatus 18, the MRI apparatus 20, and the like. Specifically, the data group includes a plurality of pieces of CT volume data, a plurality of pieces of MR volume data, and a plurality of pieces of ultrasound volume data. The data group may include volume data other than those.

Each piece of volume data is data acquired from a three-dimensional space inside a living body. Each piece of volume data is aggregate-type volume data consisting of a plurality of pieces of frame data (plurality of pieces of slice data) that are spatially connected to each other, or continuous-type volume data configured as a single piece of data with spatial continuity. Subject information, examination region information, examination date and time information, and the like are associated with each piece of data constituting the data group.

The image server 12 has a search function and an extraction function (primary extraction function). The image server 12 corresponds to a search apparatus and a primary extraction apparatus. The search function is a function of specifying one or a plurality of pieces of data satisfying a search condition in a data aggregate. The extraction function is a function of extracting frame data corresponding to a designated frame number from the volume data. The frame data is cross section data.

In the image server 12, in general, the extraction function can be applied only to the aggregate-type volume data. The designated frame number is usually a frame number corresponding to a center cross section. The image server 12 also has a function of receiving a search request and a transmit request, a function of transmitting the specific or extracted data, and the like.

The extraction server 14 is configured by an information processing apparatus such as a computer. The extraction server 14 includes an information processing unit 26 and a storage unit 28. The information processing unit 26 includes a processor that executes a program. The storage unit 28 is configured by a semiconductor memory, a hard disk, or the like. The storage unit 28 stores reference frame data 29 sent from the ultrasound diagnostic apparatus 10. The storage unit 28 also stores a program executed by the processor.

The extraction server 14 is a secondary extraction apparatus having an extraction function (secondary extraction function). Specifically, the extraction function is a function of extracting frame data (fitting frame data) that fits reference frame data from the volume data sent from the image server 12. This will be described in detail later.

The extraction server 14 also has a function of receiving the reference frame data and the volume data, a function of transmitting the fitting frame data, and the like. As will be described later, the extraction server 14 may be provided in the image server 12, or the image server 12 and the extraction server 14 may be integrated. The extraction server 14 and the ultrasound diagnostic apparatus 10 may be connected to each other by a dedicated line.

Next, the ultrasound diagnostic apparatus 10 will be described. An ultrasound probe 32 includes a transducer array. An ultrasound beam is formed by the transducer array, and electronic scanning with the ultrasound beam is performed. As an electronic scanning method, an electronic linear scanning method, an electronic sector scanning method, and the like are known. The electronic scanning with the ultrasound beam forms a beam scanning plane in the subject. The beam scanning plane is an observation cross section. A position and an orientation of the beam scanning plane in the subject change with changes in a position and an orientation of the ultrasound probe 32. A 2D transducer array may be provided in the ultrasound probe 32, and the volume data may be acquired using the 2D transducer array.

A transmission and reception circuit 34 is an electronic circuit that functions as a transmission beam former and a reception beam former. A plurality of transmission signals are output in parallel from the transmission and reception circuit 34 to the transducer array during transmission. As a result, ultrasound is radiated into the living body, that is, the transmission beam is formed. In a case where a reflected wave from an inside of the living body reaches the transducer array during reception, a plurality of reception signals are output in parallel from the transducer array to the transmission and reception circuit 34. The transmission and reception circuit 34 applies phase addition to the plurality of reception signals, thereby generating reception beam data. A plurality of pieces of reception beam data arranged in an electronic scanning direction per one electronic scanning are generated. Reception frame data is composed of the plurality of pieces of reception beam data.

A beam data processing unit 36 is a module that processes a reception beam data sequence output from the transmission and reception circuit 34. The beam data processing unit 36 includes a detector, a logarithmic converter, or the like. An image formation unit 38 generates tomographic image data (B-mode tomographic image data) based on the reception beam data sequence output from the beam data processing unit 36. The image formation unit 38 includes a digital scan converter (DSC). The DSC has a coordinate transformation function, a pixel interpolation function, or the like. The tomographic image data is sent from the image formation unit 38 to an information processing unit 40. An ultrasound image other than the tomographic image may be formed.

The information processing unit 40 includes a processor (specifically, a CPU) that executes a program. A storage unit 46 and an operation panel 48 are connected to the information processing unit 40 in addition to a display 50 described later. The storage unit 46 is configured by a semiconductor memory, a hard disk, or the like. The operation panel 48 is an input device having a plurality of switches, a track ball, a keyboard, or the like. The reference frame data may be designated or selected by the examiner by using the operation panel 48. The display 50 is configured by, for example, an organic EL display device or an LCD.

The storage unit 46 stores, for example, tissue model data 52, a plurality of pieces of tomographic image data 54, a plurality of pieces of frame data 56, a plurality of pieces of volume data 58, or the like.

The tissue model data 52 is data representing a tissue model. The tissue model is a three-dimensional model in which a form and a structure of a specific tissue (for example, a heart or a liver) are artificially simulated. A plurality of pieces of tissue model data 52 corresponding to a plurality of tissues that can be a target of the ultrasound examination may be prepared.

Each piece of tomographic image data 54 is data acquired and stored by the ultrasound diagnostic apparatus 10. The plurality of tomographic image data 54 can include current tomographic image data acquired in the current ultrasound examination on the subject and past tomographic image data acquired in the past ultrasound examination on the subject. The substance of each piece of tomographic image data 54 is frame data.

Each piece of frame data 56 is the fitting frame data extracted and transmitted by the extraction server 14. The plurality of pieces of frame data 56 can include designated frame data extracted and transmitted by the image server 12. Each piece of volume data 58 is volume data extracted and transmitted by the image server 12.

The information processing unit 40 controls an operation of each component constituting the ultrasound diagnostic apparatus 10. The information processing unit 40 functions as a background processing control unit 42 and a thumbnail image generation unit 44. The information processing unit 40 also has a function of generating a screen image displayed on the display 50. The screen image includes one or a plurality of thumbnail images representing one or a plurality of pieces of the fitting frame data in addition to the current tomographic image.

The background processing control unit 42 controls the background processing executed during the current ultrasound examination of the subject. The background processing includes search processing and thumbnail image display processing.

In the search processing, a past data set of the subject stored in the image server 12 is searched for. The past data set can include past frame data and past volume data. The past frame data is transmitted from the image server 12 to the ultrasound diagnostic apparatus 10. Optionally, the designated frame data cut out from the past volume data in accordance with the designated frame number is transmitted from the image server 12 to the ultrasound diagnostic apparatus 10 as necessary.

The past volume data specified in the image server 12 is transmitted from the image server 12 to the extraction server 14. An extraction unit 30 in the extraction server 14 extracts the fitting frame data that fits the reference frame data 29 from the received past volume data. The extracted fitting frame data is transmitted from the extraction server 14 to the ultrasound diagnostic apparatus 10. The thumbnail image generation unit 44 generates a thumbnail image based on the received or stored frame data (designated frame data, fitting frame data, or tomographic image data).

In the above-described thumbnail image display processing, the thumbnail image representing the past data stored in the image server 12 is displayed on the display 50. In that case, the thumbnail image represents the past frame data or the past volume data obtained from the above-described subject. A thumbnail image representing the tomographic image data acquired and stored in the current ultrasound examination is also displayed on the display 50.

It should be noted that the thumbnail image representing the data (external data) stored in the image server 12 is a first thumbnail image, and the thumbnail image representing the data (internal data) stored in the ultrasound diagnostic apparatus 10 is a second thumbnail image. The thumbnail image representing the designated frame data extracted from the volume data stored in the image server 12 is a primary thumbnail image. The thumbnail image representing the fitting frame data extracted from the volume data stored in the image server 12 is a secondary thumbnail image. Both the primary thumbnail image and the secondary thumbnail image are the first thumbnail image. The screen image displayed on the display 50 includes one or a plurality of first thumbnail images and one or a plurality of second thumbnail images.

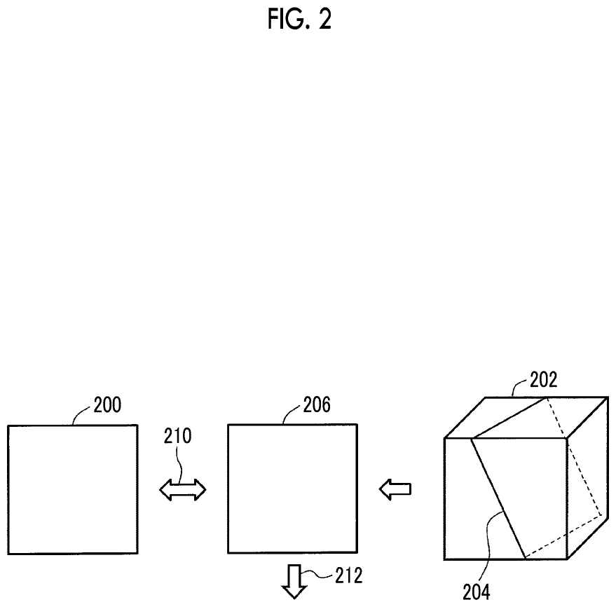

FIG. 2 shows the extraction processing of the fitting frame data in the extraction server 14. In general, a plurality of cutout cross sections 204 are sequentially set at various positions and various angles with respect to received volume data 202. A reference numeral 206 indicates candidate frame data corresponding to the specific cutout cross section 204. Each piece of sequentially extracted candidate frame data 206 is compared with reference frame data 200 (refer to a reference numeral 210). In such a case, a technique such as a correlation calculation or pattern matching is used.

A fitting score is calculated for each comparison of the frame data. The fitting score is, for example, a correlation value, a matching score, or a similarity. A best fitting score is specified from among the plurality of fitting scores corresponding to the plurality of pieces of candidate frame data, and then the candidate frame data corresponding to the best fitting score is specified as the fitting frame data. As a result, the fitting frame data is extracted from the volume data. The fitting frame data is sent from the extraction server 14 to the ultrasound diagnostic apparatus 10 together with score information indicating the fitting score corresponding to the fitting frame data (refer to a reference numeral 212). The fitting frame data and the score information may be sent to the ultrasound diagnostic apparatus 10 via the image server 12. It should be noted that, in a case of comparing two pieces of frame data, a size or a scale of either piece of frame data is adjusted as necessary.

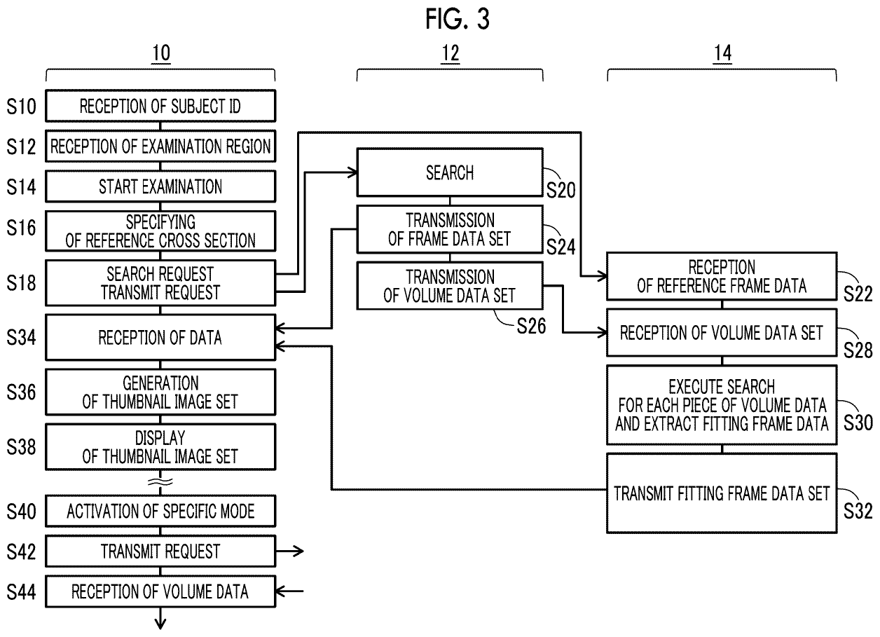

FIG. 3 shows an operation of the ultrasound diagnostic system shown in FIG. 1. More specifically, FIG. 3 shows the operation of the ultrasound diagnostic apparatus 10, the operation of the image server 12, and the operation of the extraction server 14 in the background processing.

In the ultrasound diagnostic apparatus 10, in S10, subject ID is received, and in S12, an examination region is received. The examination region is a region to which an examination target tissue belongs (for example, abdomen or circulatory system). In S14, the ultrasound examination is started. In S16, the reference cross section is specified. That is, the reference frame data is specified. The reference frame data may be automatically specified, or the reference frame data may be designated by the examiner.

In S18, the search request and the transmit request are sent from the ultrasound diagnostic apparatus 10 to the image server 12. The search request may be sent in advance, and the transmit request based on a search result may be sent after the search result is received. The search request and the transmit request may be simultaneously sent. The search request includes the search condition. The search condition includes subject identification information (subject ID), examination region identification information, a search period, and the like. The transmit request includes the reference frame data. The transmit request includes designated frame identification information as necessary. The reference frame data is sent from the ultrasound diagnostic apparatus 10 to the extraction server 14. The reference frame data may be sent from the ultrasound diagnostic apparatus 10 to the extraction server 14 via the image server 12. In S22, the extraction server 14 receives the reference frame data.

In the image server 12, in S20, the data set satisfying the search condition is searched for from the stored data group. The data set includes one or a plurality of pieces of frame data (frame data set) and/or one or a plurality of pieces of volume data (volume data set). Any piece of the data is past data obtained from the subject who is a target of the current ultrasound examination.

In a case where the frame data set is specified by the above-described search, the frame data set is transmitted from the image server 12 to the ultrasound diagnostic apparatus 10 in S24. In this case, the designated frame data extracted from each piece of volume data is also transmitted as necessary. In addition, in a case where the volume data set is specified by the above-described search, the volume data set is transmitted from the image server 12 to the extraction server 14 in S26.

In the extraction server 14, in S28, the volume data set is received. In S30, for each piece of volume data, the candidate frame data that fits the reference frame data is searched for from within the volume data. In S32, a fitting frame data set extracted from the volume data set is transmitted from the extraction server 14 to the ultrasound diagnostic apparatus 10. The fitting frame data set may be transmitted from the extraction server 14 to the ultrasound diagnostic apparatus 10 via the image server 12.

In the ultrasound diagnostic apparatus 10, in S34, the frame data set sent from the image server 12 is received, and the fitting frame data set sent from the extraction server 14 is received. Any piece of the received data is the past data acquired from the subject as described above. In S36, a plurality of thumbnail images (thumbnail image set) are generated based on the received plurality of pieces of data. In S38, the thumbnail image set is displayed. In general, a plurality of the thumbnail images are sequentially displayed in a generation order thereof.

The displayed thumbnail image set includes the thumbnail image representing the fitting frame data extracted from the volume data. The examiner can recognize that the past volume data is present in the image server 12 through observation of such a thumbnail image.

For example, after the activation of a two-screen display mode, the current tomographic image as a real-time tomographic image and the past tomographic image generated from the fitting frame data may be displayed in juxtaposition through a selection operation of the thumbnail image. In that case, it is possible to evaluate content of the current tomographic image while comparing content of the two images. The reference frame data is selected in advance in order to appropriately perform such image comparison.

In the operation example shown in FIG. 3, in S40, a specific mode is activated during the current ultrasound examination. After that, in S42, a transmit request for the volume data corresponding to the thumbnail image is sent to the image server 12 through the selection operation of the thumbnail image. In S44, the volume data sent from the image server 12 is received by the ultrasound diagnostic apparatus 10.

The specific mode is, for example, a mode in which the current tomographic image as the real-time tomographic image and the past tomographic image based on the past volume data are displayed in juxtaposition. In that case, both the current tomographic image and the past tomographic image are images representing the same observation cross section (beam scanning plane). For example, based on position information obtained by detecting the position and the orientation of the ultrasound probe 32, plane data corresponding to the observation cross section is cut out from the past volume data, and the past tomographic image is generated based on the plane data.

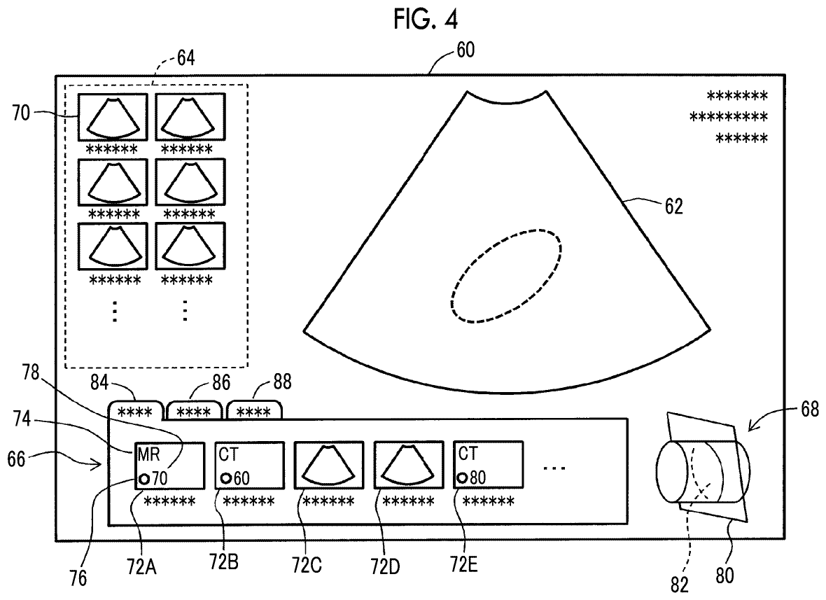

FIG. 4 shows an example of a screen image displayed on the display 50. The screen image 60 includes a real-time tomographic image 62, a first thumbnail image display area 64, and a second thumbnail image display area 66.

The first thumbnail image display area 64 is an area for displaying a plurality of thumbnail images 70 representing a plurality of pieces of internal data. Each piece of internal data is data acquired and stored in the ultrasound diagnostic apparatus 10, and is typically tomographic image data. The second thumbnail image display area 66 is an area for displaying a plurality of thumbnail images 72A to 72E representing a plurality of pieces of external data. Each piece of the external data is data stored in the image server 12, and is the frame data or the volume data.

The thumbnail image 72A is a thumbnail image representing the MR volume data acquired by the MRI apparatus. The thumbnail image 72A includes a label 74 indicating a data type, a mark 76 indicating that the image is the thumbnail image representing the fitting frame data, and a numerical value 78 indicating the fitting score. Although the mark 76 is a simple circular mark, various display elements can be adopted as the mark 76. A maximum value of the numerical value 78 is, for example, 100 (%), and a minimum value thereof is, for example, 0 (%). Instead of the numerical value, information such as a signal and a color may be displayed.

By displaying the mark 76, the examiner can recognize that the thumbnail image 72A is the thumbnail image representing the fitting frame data. By displaying the numerical value 78, the examiner can be presented with the possibility that the fitting frame data is an image representing a cross section that the examiner would like to see.

In the second thumbnail image display area 66, a window selected from among a plurality of windows is actually displayed. A specific tab is selected from among a plurality of tabs 84, 86, and 88 corresponding to the plurality of windows, and a window corresponding to the selected tab is displayed. One or the plurality of the second thumbnail images are included in each individual window. For example, the window may be generated for each examination date or for each examination period.

The screen image shown in FIG. 4 includes a tissue model image 68. The tissue model image 68 is an image representing a three-dimensional model obtained by simulating a tissue that is a target of the current ultrasound examination. A frame image 80 that crosses the tissue model image 68 is also displayed. A position and an orientation of the frame image 80 with respect to the tissue model image 68 are adjusted by the examiner. As a result, a specific cross section 82 in the tissue model image 68 is specified. Based on the specific cross section 82, the cross section data is cut out from the tissue model. The frame data is used as the reference frame data. As will be described later, the reference frame data may be defined based on other data.

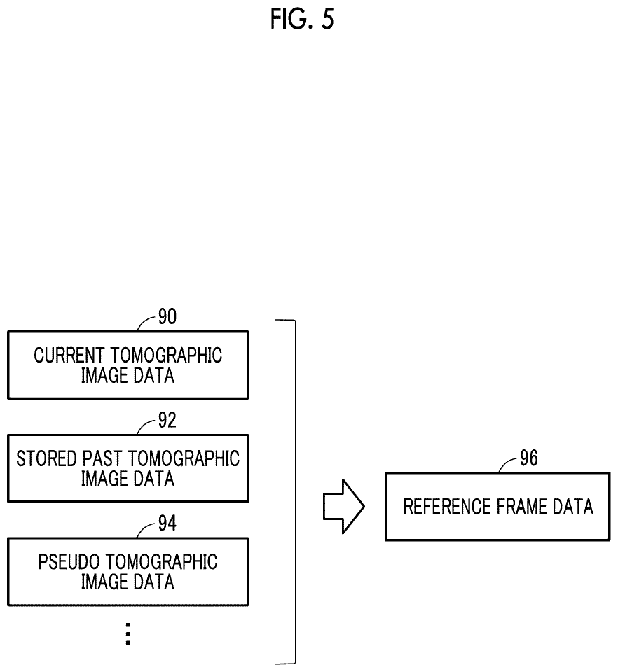

FIG. 5 shows a determination method of reference frame data 96. For example, the current tomographic image data 90 may be the reference frame data 96. In that case, the current tomographic image data 90 may be specified at a timing designated by the examiner or at a timing automatically determined. The current tomographic image at the freezing point in time may be used as the reference frame data.

Past tomographic image data 92 stored in the ultrasound diagnostic apparatus 10 may be used as the reference frame data 96. The past tomographic image data 92 is past data acquired from the subject who is currently undergoing the ultrasound examination. As described above, pseudo tomographic image data 94, which is the cross section data cut out from the three-dimensional tissue model, may be the reference frame data 96.

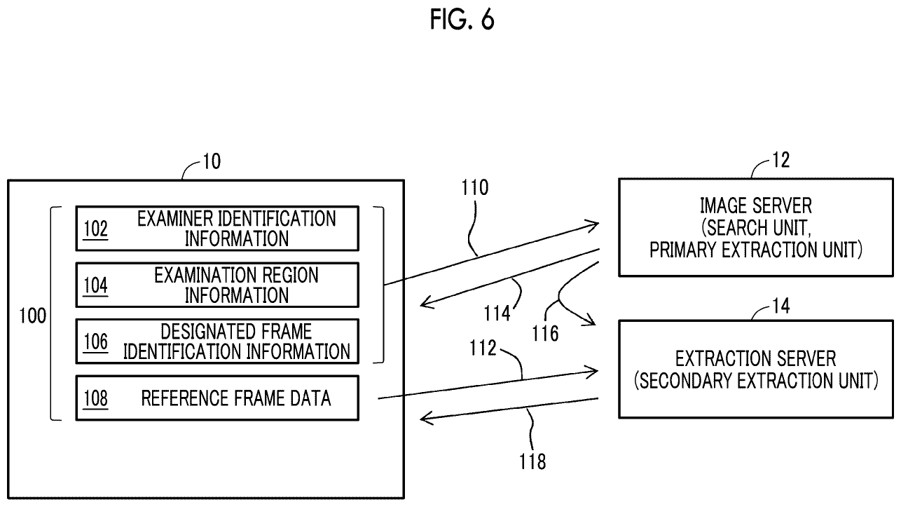

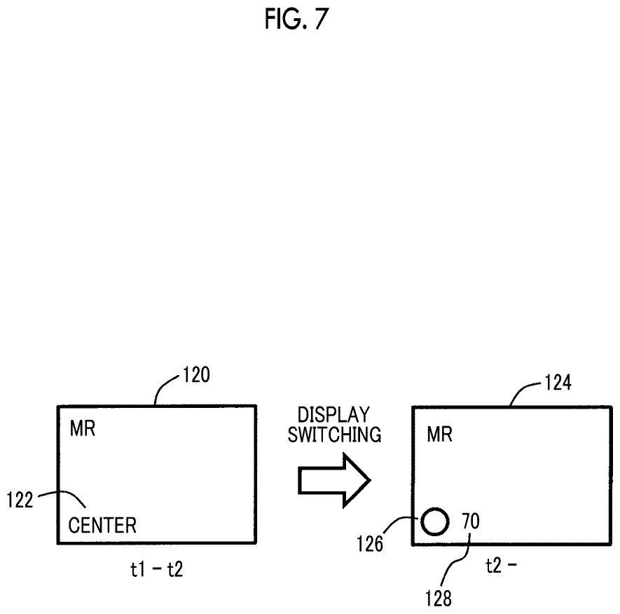

Hereinafter, an ultrasound diagnostic system according to a second embodiment will be described with reference to FIGS. 6 and 7. A configuration of an ultrasound diagnostic system according to the second embodiment is basically the same as the configuration shown in FIG. 1. In the second embodiment, the primary thumbnail image based on the designated frame data is displayed first, and then the secondary thumbnail image based on the fitting frame data is displayed. Hereinafter, the present embodiment will be specifically described.

A reference numeral 100 indicates information included in the search request (and the transmit request) issued from the ultrasound diagnostic apparatus 10. In the search request 100, examiner identification information 102, examination region information 104, and designated frame identification information 106 are sent to the image server 12 (refer to a reference numeral 110). The search condition is composed of the examiner identification information 102 and the examination region information 104. The search condition may include other conditions, such as a search period.

The designated frame identification information 106 specifies a frame number of a frame to be extracted from the volume data. In general, the center (middle) frame number is specified based on a head frame number and a last frame number, and the center frame number is the designated frame identification information 106. In the search request 100, reference frame data 108 is sent to the extraction server 14 (refer to a reference numeral 112).

The image server 12 specifies a data set satisfying the search condition from among the data group stored therein. From that viewpoint, the image server 12 is a search unit or a search apparatus. In a case where one or a plurality of pieces of volume data are included in the specified data set, the image server 12 extracts the designated frame data specified by the designated frame identification information from the volume data for each piece of volume data. The processing is primary extraction processing, and the image server 12 is a primary extraction unit or a primary extraction apparatus from that viewpoint. The image server 12 transmits the frame data set to the ultrasound diagnostic apparatus 10 (refer to a reference numeral 114). In the second embodiment, the frame data set includes one or a plurality of pieces of designated frame data.

On the other hand, in a case where the volume data set is specified by the search in the image server 12, the volume data set is sent from the image server 12 to the extraction server 14 (refer to a reference numeral 116) in parallel with the above-described designated frame data extraction processing. The extraction server 14 extracts the fitting frame data that fits the reference frame data from the volume data for each piece of volume data. The processing is secondary extraction processing, and from that viewpoint, the extraction server 14 is a secondary extraction unit or a secondary extraction apparatus. The extraction server 14 transmits the fitting frame data set extracted from the volume data set to the ultrasound diagnostic apparatus 10 (refer to a reference numeral 118).

The ultrasound diagnostic apparatus 10 generates a plurality of thumbnail images based on the frame data set (including a designated frame data set) sent from the image server 12 and the fitting frame data set sent from the extraction server 14.

In general, it takes a certain time to extract the fitting frame data. On the other hand, the designated frame data can be quickly extracted. In consideration of this, the ultrasound diagnostic apparatus 10 generates the primary thumbnail image based on the designated frame data at a point in time of receiving the designated frame data extracted from the volume data, and displays the primary thumbnail image in advance. Thereafter, the ultrasound diagnostic apparatus 10 generates the secondary thumbnail image based on the fitting frame data at a point in time of receiving the fitting frame data extracted from the same volume data, and displays the secondary thumbnail image instead of the primary thumbnail image.

With the above-described switching of thumbnail images, it is possible to avoid an increase in a blank time in which a thumbnail image is not displayed, and it is possible to satisfy a need to display a thumbnail image that the examiner would like to observe.

FIG. 7 shows an example of the switching of thumbnail images. First, a primary thumbnail image 120 based on the designated frame data extracted from the specific volume data is displayed. Subsequently, a secondary thumbnail image 124 based on the fitting frame data extracted from the same specific volume data is displayed instead of the primary thumbnail image 120.

The primary thumbnail image 120 includes information 122 for specifying the frame number or a position of the designated frame. In the shown example, the information 122 indicates the middle (center) of the volume data. The primary thumbnail image is displayed over a period from t1 to t2.

The secondary thumbnail image 124 includes a mark 126 for identifying that the displayed thumbnail image is the secondary thumbnail image. In a case where the mark 126 is not displayed, it can be determined that the displayed thumbnail image is the primary thumbnail image. The numerical value 128 is information representing the fitting score. The secondary thumbnail image 124 is displayed in a period after t2.

For each piece of individual volume data, individual display switching of thumbnail images may be executed at a point in time at which the generation of the secondary thumbnail image is completed. In that case, the display start of a large number of secondary thumbnail images can be accelerated. On the other hand, the display switching of the plurality of thumbnail images may be collectively executed at a point in time at which the generation of a plurality of secondary thumbnail images corresponding to the plurality of pieces of volume data constituting the volume data set is completed. In that case, observation hindrance due to the repeated display switching can be prevented.

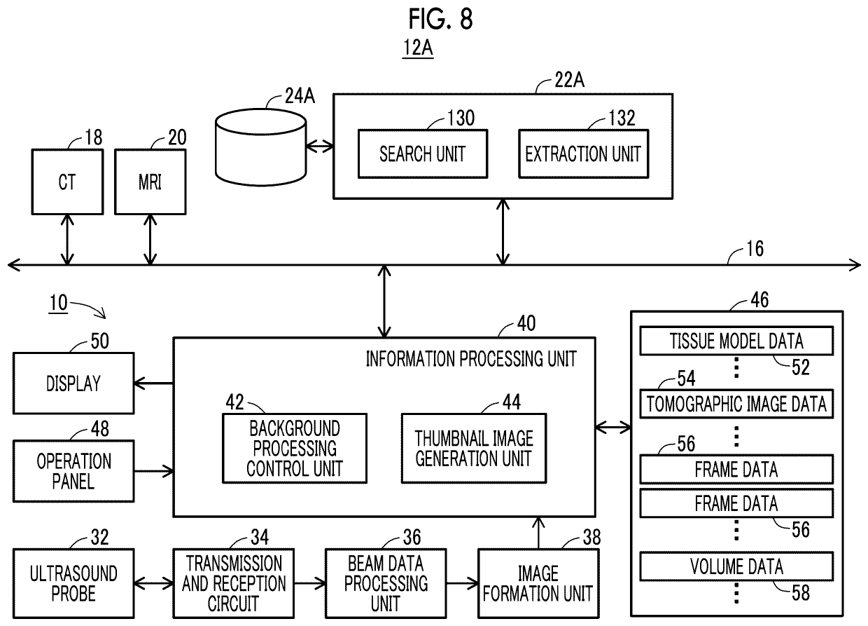

FIG. 8 shows an ultrasound diagnostic system according to a third embodiment. In FIG. 8, the same reference numerals are added to the same components as the components shown in FIG. 1.

In the third embodiment, an image server 12A includes an information processing unit 22A. The information processing unit 22A functions as a search unit 130 and an extraction unit 132. The extraction unit 132 is a module that executes the primary extraction processing and the secondary extraction processing. That is, the image server 12A is a server including the extraction server shown in FIG. 1. In addition to the data group, the reference frame data is stored in a storage unit 24A.

The extraction apparatus shown in FIG. 1 may be provided in an external information processing apparatus that is physically coupled to the ultrasound diagnostic apparatus 10. In this case, the ultrasound diagnostic apparatus 10 and the external information processing apparatus may be connected to each other by a dedicated line.

What is claimed is:

1. An ultrasound diagnostic system comprising:
an ultrasound diagnostic apparatus configured to form an ultrasound image based on reception data obtained from a subject; and
an extraction apparatus configured to extract, in a case where past volume data obtained from the subject is specified from a data group stored in a server, fitting frame data that fits reference frame data sent from the ultrasound diagnostic apparatus from the past volume data,
wherein the ultrasound diagnostic apparatus displays an image representing the fitting frame data together with the ultrasound image during an ultrasound examination of the subject.

2. The ultrasound diagnostic system according to claim 1,
wherein the extraction apparatus compares the reference frame data with a plurality of pieces of candidate frame data included in the past volume data to extract specific candidate frame data as the fitting frame data.

3. The ultrasound diagnostic system according to claim 1,
wherein the ultrasound diagnostic apparatus includes a thumbnail image generation unit that generates a thumbnail image as the image representing the fitting frame data, based on the fitting frame data.

4. The ultrasound diagnostic system according to claim 3,
wherein the ultrasound diagnostic apparatus displays a screen image including the ultrasound image,
the thumbnail image is a first thumbnail image,
the thumbnail image generation unit further generates a second thumbnail image representing the reception data obtained by the ultrasound diagnostic apparatus, and
the screen image includes
an external data display area in which the first thumbnail image is displayed, and
an internal data display area in which the second thumbnail image is displayed.

5. The ultrasound diagnostic system according to claim 3,
wherein the extraction apparatus is a secondary extraction apparatus,
the thumbnail image is a secondary thumbnail image,
a primary extraction apparatus that extracts designated frame data from the past volume data based on designated frame identification information sent from the ultrasound diagnostic apparatus is provided,
the thumbnail image generation unit further generates a primary thumbnail image as an image representing the designated frame data, based on the designated frame data, and
the ultrasound diagnostic apparatus displays the primary thumbnail image and the secondary thumbnail image.

6. The ultrasound diagnostic system according to claim 5,
wherein the ultrasound diagnostic apparatus displays the primary thumbnail image first, and then displays the secondary thumbnail image instead of the primary thumbnail image.

7. The ultrasound diagnostic system according to claim 5,
wherein the ultrasound diagnostic apparatus displays identification information for identifying the primary thumbnail image and the secondary thumbnail image.

8. The ultrasound diagnostic system according to claim 2,
wherein the extraction apparatus calculates a fitting score in a case of comparing the reference frame data and the candidate frame data, and
the ultrasound diagnostic apparatus displays information indicating a fitting score corresponding to the fitting frame data together with the image representing the fitting frame data.

9. The ultrasound diagnostic system according to claim 1,
wherein the reference frame data is one of past tomographic image data registered in the ultrasound diagnostic apparatus, current tomographic image data acquired in a current ultrasound examination on the subject, or pseudo tomographic image data cut out from a three-dimensional tissue model.

10. An extraction apparatus comprising:
means for receiving reference frame data sent from an ultrasound diagnostic apparatus;
means for extracting, in a case where past volume data obtained from a subject is specified from a data group stored in a server, fitting frame data that fits the reference frame data from the past volume data; and
means for transmitting the fitting frame data to the ultrasound diagnostic apparatus,
wherein an image representing the fitting frame data is displayed together with an ultrasound image during an ultrasound examination of the subject, in the ultrasound diagnostic apparatus.

11. A non-transitory storage medium storing a program executed in an information processing apparatus, the program including:
a function of receiving reference frame data sent from an ultrasound diagnostic apparatus;
a function of extracting, in a case where past volume data obtained from a subject is specified from a data group stored in a server, fitting frame data that fits the reference frame data from the past volume data; and
a function of transmitting the fitting frame data to the ultrasound diagnostic apparatus,
wherein an image representing the fitting frame data is displayed together with an ultrasound image during an ultrasound examination of the subject, in the ultrasound diagnostic apparatus.
